(19) United States
(12) Patent Application Publication
(10) Pub. No.: US 2026/0007878 A1
(43) Pub. Date: Jan. 8, 2026

(54) TRANSIENT ELECTRODES AND ASSOCIATED SYSTEMS AND METHODS FOR NEURAL MODULATION AND RECORDING

(71) Applicant: Case Western Reserve University, Cleveland, OH (US)

(72) Inventors: Andrew SHOFFSTALL, Cleveland, OH (US); Derrick LIU, Cleveland, OH (US); Danny LAM, Cleveland, OH (US); Laurie DUDIK, Cleveland, OH (US); Allison HESS-DUNNING, Cleveland, OH (US); Anna LAURICELLA, Cleveland, OH (US)

(21) Appl. No.: 18/992,764

(22) PCT Filed: Jul. 12, 2023
(86) PCT No.: PCT/US2023/027496

§ 371 Date: Jan. 9, 2025

Related U.S. Application Data
(60) Provisional application No. 63/388,264, filed on Jul. 12, 2022.
(60) Provisional application No. 63/434,884, filed on Dec. 22, 2022.

(51) Int. Cl. A61N 1/05; A61N 1/36; A61N 1/372

(52) U.S. Cl. CPC A61N 1/0551; A61N 1/0502; A61N 1/36017; A61N 1/36071; A61N 1/36103; A61N 1/36146; A61N 1/37217

(57) ABSTRACT

The present disclosure relates generally to devices, systems and methods for neuromodulation and neural recording and, in particular, to transient electrodes and associated systems and methods for neuromodulation and neural recording.

### RELATED APPLICATIONS

This application claims the benefit of U.S. Provisional Patent Application Ser. No. 63/388,264, filed Jul. 12, 2022, and also claims the benefit of U.S. Provisional Patent Application Ser. No. 63/434,884, filed Dec. 22, 2022. The entirety of each of these provisional patent applications is hereby incorporated by reference for all purposes.

### TECHNICAL FIELD

The present disclosure relates generally to devices, systems and methods for neuromodulation and neural recording and, in particular, to transient electrodes and associated systems and methods for neuromodulation and neural recording.

### BACKGROUND

Electrical stimulation after peripheral nerve surgical resection, transection, or other physical trauma has been shown to enhance nerve regeneration and lead to better functional outcomes (e.g., reduced pain, incidence of neuroma, and restoration of nerve function). However, permanent indwelling electrodes form a fibrous capsule that makes removal at later time points difficult. For example, removal of such devices can lead to re-injury of the nerve or, as is common, fractured devices that then remain within the body.

### SUMMARY

The present disclosure relates generally to devices, systems and methods for neuromodulation and neural recording and, in particular, to transient electrodes and associated systems and methods for neuromodulation and neural recording.

One aspect of the present disclosure can include a transient electrode comprising: an electrically non-conductive core substrate; a continuous, electrically-conductive metal layer that envelops at least a portion of the core substrate; an optional interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a transient electrode comprising: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a transient electrode comprising: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and a barrier layer that at least partially envelops the metal layer; wherein the entire electrode biodegrades and is rendered non-functional after a period of time in vivo.

Another aspect of the present disclosure can include a system comprising: a transient electrode; and a power source in electrical communication with the transient electrode; wherein the transient electrode comprises: an electrically non-conductive core substrate; a continuous, electrically-conductive metal layer that envelops at least a portion of the core substrate; an optional interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a system comprising: a transient electrode; and a power source in electrical communication with the transient electrode; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a system comprising: a transient electrode; and a power source in electrical communication with the transient electrode; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and a barrier layer that at least partially envelops the metal layer; wherein the entire electrode biodegrades and is rendered non-functional after a period of time in vivo.

Another aspect of the present disclosure can include a method for temporary neuromodulation of a target nervous tissue in a subject in need thereof, the method comprising: advancing a transient electrode into electrical contact with the target nervous tissue; and delivering a therapy signal to the target nervous tissue via the transient electrode for a period of time until the transient electrode completely biodegrades and is rendered non-functional; wherein the transient electrode comprises: an electrically non-conductive core substrate; a continuous, electrically-conductive metal layer that envelops at least a portion of the core substrate; an optional interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a method for temporary neuromodulation of a target nervous tissue in a subject in need thereof, the method comprising: advancing a transient electrode into electrical contact with the target nervous tissue; and delivering a therapy signal to the target nervous tissue via the transient electrode for a period of time until the transient electrode completely biodegrades and is rendered non-functional; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a method for temporary neuromodulation of a target nervous tissue in a subject in need thereof, the method comprising: advancing a transient electrode into electrical contact with the target nervous tissue; and delivering a therapy signal to the target nervous tissue via the transient electrode for a period of time until the transient electrode completely biodegrades and is rendered non-functional; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and a barrier layer that at least partially envelops the metal layer; wherein the entire electrode biodegrades and is rendered non-functional after a period of time in vivo.

Another aspect of the present disclosure can include a method for temporary recording of electrical activity in a target nervous tissue in a subject, the method comprising: advancing a transient electrode into electrical contact with the target nervous tissue; and recording, by the transient electrode, the electrical activity of the target nervous tissue for a period of time until the transient electrode completely biodegrades and is rendered non-functional; wherein the transient electrode comprises: an electrically non-conductive core substrate; a continuous, electrically-conductive metal layer that envelops at least a portion of the core substrate; an optional interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a method for temporary recording of electrical activity in a target nervous tissue in a subject, the method comprising: advancing a transient electrode into electrical contact with the target nervous tissue; and recording, by the transient electrode, the electrical activity of the target nervous tissue for a period of time until the transient electrode completely biodegrades and is rendered non-functional; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Another aspect of the present disclosure can include a method for temporary recording of electrical activity in a target nervous tissue in a subject, the method comprising: advancing a transient electrode into electrical contact with the target nervous tissue; and recording, by the transient electrode, the electrical activity of the target nervous tissue for a period of time until the transient electrode completely biodegrades and is rendered non-functional; wherein the transient electrode comprises: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and a barrier layer that at least partially envelops the metal layer; wherein the entire electrode biodegrades and is rendered non-functional after a period of time in vivo.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a schematic illustration showing a system comprising a transient electrode in electrical communication with a power source according to one aspect of the present disclosure;

FIG. 1B is a cross-sectional view of the transient electrode in FIG. 1A taken along Line 1B-1B;

FIG. 1C is a schematic illustration showing a cutaway view of the transient electrode in FIG. 1A;

FIG. 2A is a schematic illustration showing a system comprising a transient electrode in electrical communication with a power source according to another aspect of the present disclosure;

FIG. 2B is a cross-sectional view of the transient electrode in FIG. 2A taken along Line 2B-2B;

FIG. 3A is a schematic illustration showing a system comprising a transient electrode in electrical communication with a power source according to yet another aspect of the present disclosure;

FIG. 3B is a cross-sectional view of the transient electrode in FIG. 3A taken along Line 3B-3B;

FIG. 4 is a schematic illustration showing alternative constructions of the transient electrode in FIGS. 1A-3B;

FIG. 5 is a schematic illustration showing various approaches for implanting the transient electrode of FIGS. 1A-3B in a subject;

FIG. 6 is a schematic illustration showing a fabrication scheme for the transient electrode in FIGS. 1A-B;

FIG. 7 is a series of images showing a microcracked, electrically-conductive metal (gold) layer of a transient electrode, under digital and scanning electron microscopy, according to one example of the present disclosure;

FIG. 8 is a Bode plot (averaged, n=8) showing baseline EIS impedance (blue) and phase (red) behavior of a transient electrode in 1×PBS, according to one example of the present disclosure;

FIG. 9 is a Cyclic Voltammogram (averaged, n=8) showing baseline I (current)-V (Potential vs. Ag/AgCl) characteristics of a transient electrode in 1×PBS, according to one example of the present disclosure;

FIG. 10 is a plot showing Corresponding Cathodic Charge Storage Capacity of a transient electrode in 20 mM H₂O₂/PBS reactive aging solution over time (4 weeks), according to one example of the present disclosure;

FIG. 11 is a microscopy image showing swelling and delamination along the length of a transient electrode after 14 days in 20 mM H₂O₂/PBS reactive aging solution, according to one example of the present disclosure;

FIG. 12 is a plot showing impedance (n=6) EIS at 1 KHz of a transient electrode in 20 mM H₂O₂/PBS reactive aging solution over time (4 weeks), according to one example of the present disclosure; and

FIG. 13 is a plot showing percent survival of subcutaneously implanted transient electrodes over 14 days (n=7), according to one example of the present disclosure.

### DETAILED DESCRIPTION

### Definitions

Unless defined otherwise, all technical and scientific terms used herein have the same meaning as is commonly understood by one of skill in the art to which the present disclosure pertains.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In the context of the present disclosure, the term “about”, when expressed as from “about” one particular value and/or “about” another particular value, also specifically contemplated and disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these aspects are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

As used herein, phrases such as “between X and Y” and “between about X and Y” can be interpreted to include X and Y.

As used herein, phrases such as “between about X and Y” can mean “between about X and about Y”.

As used herein, phrases such as “from about X to Y” can mean “from about X to about Y”.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “optionally” and “optional” can mean that the subsequently described event, circumstance, or material may or may not occur or be present, and that the description includes instances where the event, circumstance, or material occurs or is present and instances where it does not occur or is not present.

As used herein, the terms “biodegradation” or “biodegrades”, when referring to a transient electrode of the present disclosure, can mean degradation of the material(s) comprising the transient electrode in vivo, whereafter clearance of the degraded material(s) from surrounding biological tissue occurs based primarily on diffusion and active transport mechanisms by cells to remove debris through excretion. In other words, material(s) from the degraded transient electrode are not locally resorbed; rather, such material(s) is/are cleared through diffusion and active transport thereof. For example, gold is not naturally used in biological processes and will not be locally resorbed; rather, it will be cleared through diffusion and active transport.

As used herein, the term “bioresorbable” can refer to degradation of material(s) in vivo, whereafter the degraded material(s) is/are locally cleared from surrounding biological tissues by resorption. For example, degraded materials such as magnesium and iron can be taken up and utilized in biological processes by cells comprising the biological tissue.

As used herein, the term “continuous”, when referring to an electrically-conductive metal layer of the present disclosure, can mean a layer of an electrically-conductive metal that is uniform and has a conformed and/or predetermined thickness over an area of a non-conductive core substrate or an interlayer of a transient electrode of the present disclosure. Further a continuous metal layer can mean that the metal layer is not a network of discrete, electrically-conductive particles. Percolation of electrically-conductive particles held by a polymer matrix relies on proximity of the particles to one another to maintain bulk electrical conductivity. This network may be disrupted by polymer swelling and water that surrounds and permeates the polymer structure over time. A continuous metal layer of the present disclosure, in contrast, advantageously allows for metal bonding, forms a barrier to water permeation, and is less susceptible to swelling and rapid breakdown of bulk electrical conductivity as compared to a conductive coating comprising a network of discrete, electrically-conductive particles.

As used herein, the term “electrical communication” can refer to the ability of a generated electric field to be transferred to, or have an effect on, one or more components of the present disclosure. In some instances, a generated electric field can be directly transferred to a component (e.g., via a wire or lead). In other instances, a generated electric field can be wirelessly transferred to a component. In one example, the term “electrical communication” can refer to the ability of an electric field to be transferred to, or have a neuromodulatory effect, within and/or on at least one target nervous tissue, neuron, and/or nervous tissue of a subject.

As used herein, the terms “microcrack” or “microcracked”, when referring to a metal layer as disclosed herein, can mean a microsurface of the metal layer characterized as having a plurality of layers, each of which includes a series of incomplete fractures. Advantageously, a microcracked metal layer of the present disclosure: (1) improves the surface area of the transient electrode, which improves electrical conductivity thereof; and (2) and lead to improved degradation in vivo as this creates an opportunity for water ingress into the transient electrode, thereby leading to delamination of the metal layer and interaction with the core substrate that further leads to its degradation in vivo.

As used herein, the term “target nervous tissue” can refer to a cell, nerve, or neural cell comprising the nervous system of an animal (including a human) to which a therapy signal is applied via a transient electrode of the present disclosure. The term can encompass single cells as well as an aggregate of cells that can be part of, or associated with, a neuron or nerve of the peripheral nervous system (PNS). A “nerve” can refer to a bundle of nerve fibers enclosed by a nerve sheath. In one example, “target nervous tissue” can comprise a peripheral nerve; that is, a number of fibers of either the somatic or autonomic nervous system, which are not part of the CNS.

As used herein, the term “therapy signal” can refer to an electrical signal, having desired characteristics (e.g., voltage, pulse-width, frequency), that is delivered to a target nervous tissue and is capable of modulating (e.g., electrically modulating) the target nervous tissue to effect a change in the target nervous tissue.

As used herein, the terms “modulate” or “modulating”, with reference to application of a therapy signal to a target nervous tissue, can refer to causing a change in neuronal activity, chemistry, and/or metabolism of the target nervous tissue. The change can refer to an increase, decrease, or even a change in a pattern of neuronal activity. The terms may refer to either excitatory stimulation (activation) or application of electrical energy that entirely or partially inhibits or blocks nerve activity (e.g., conduction), or a combination thereof. The terms can also be used to refer to a masking, altering, overriding, or restoring of neuronal activity.

As used herein, the term “transient”, when referring to a transient electrode of the present disclosure, can mean impermanent or persisting for only a limited period of time. The term may also be used interchangeably with “temporary” herein.

As used herein, the term “pure”, when referring to a metal described herein, can mean a metal having a high purity, such as about 99% or greater purity, about 99.5% or greater purity, about 99.9% or greater purity, or about 99.99% or greater purity. As can be appreciated, purity can alternatively be measured using alternative notation systems. For example, in some instances, suitable metals can be 4N or 5N pure, which refer to metals having 99.99% and 99.999% purity, respectively. As used herein, “purity” can refer to either absolute purity or metal basis purity in certain aspects.

As used herein, the term “non-functional”, when referring to a transient electrode as described herein, can mean incapable of conducting an electrical charge; that is, having an electrical conductivity (σ) of zero.

As used herein, the term “subject” can refer to a vertebrate, such as a mammal (e.g., a human). Mammals can include, but are not limited to, humans, dogs, cats, horses, cows, and pigs.

### Overview

Conventional neuromodulation technologies are constrained by the need for ease-of-removal versus the requirement for a robust-nerve-interface. In the case of patients having undergone total knee replacement (TKA), for example, sensory nerves of the knee (lateral superior, lateral inferior and recurrent genicular nerves) are commonly targeted for RF thermal ablation following TKA. Due to the transiency of post-operative pain, fully implanted permanent systems are not necessary and raise costs and risks for neuromodulation. Percutaneous systems may be a viable alternative; however, such systems suffer from several drawbacks, including increased risk for infection due to exit sites of the percutaneous leads. Furthermore, percutaneous leads are consistently noted by patients during clinical studies as a nuisance (requiring additional care and discomfort).

Advantageously, the inventors of the present disclosure have developed transient electrodes and related systems and methods that obviate the need for conventional percutaneous wires and permanent implants based, at least in part, on the unexpected discovery of a transient electrode that, upon implantation in a subject, can deliver a therapy signal to a target nervous tissue and then, after a limited period of time, completely biodegrade in vivo so as to render the electrode non-functional.

### Transient Electrodes

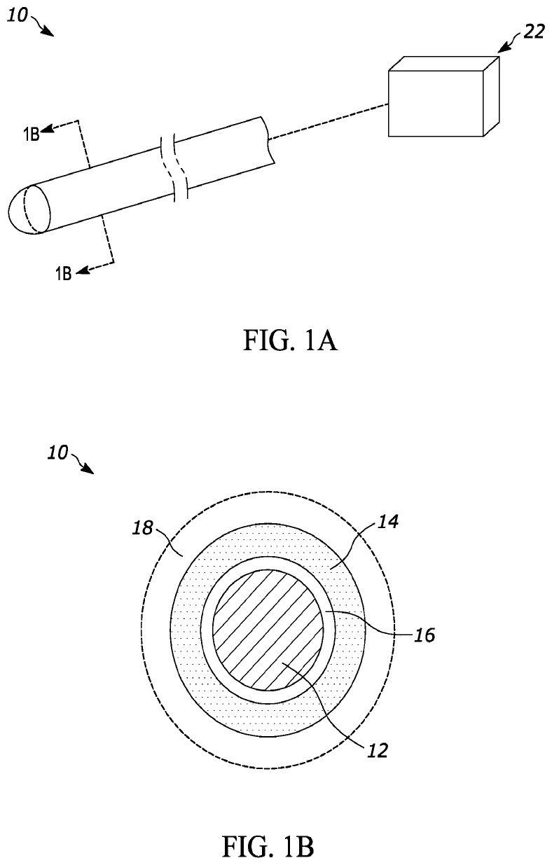

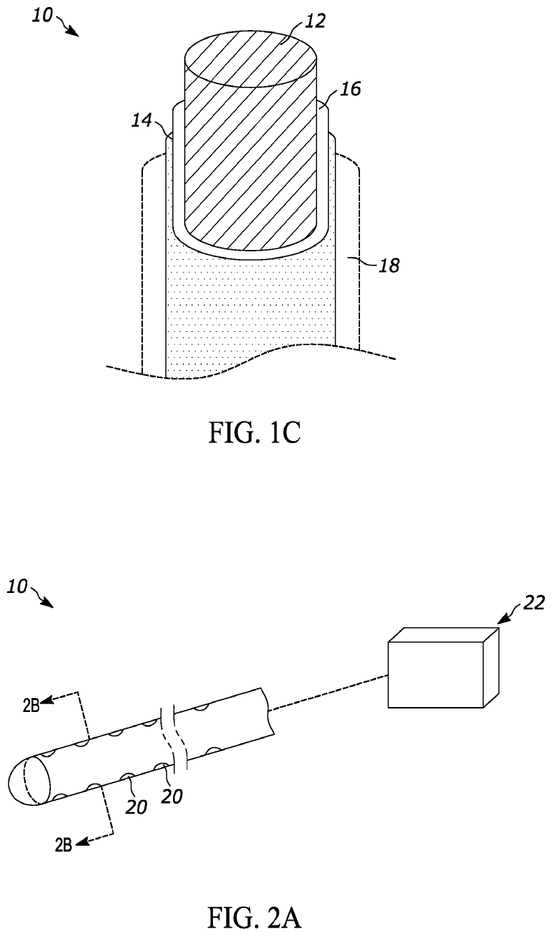

One aspect of the present disclosure can include a transient electrode 10 (FIGS. 1A-C) that biodegrades so as to render the transient electrode non-functional after a period of time in vivo.

In one embodiment, the transient electrode 10 biodegrades at least about 50%, at least about 55%, at least about 60%, at least about 65%, at least about 70%, at least about 75%, at least about 80%, at least about 85%, at least about 90%, at least about 95%, at least about 99%, or 100% (completely) so as to render the transient electrode non-functional after a period of time in vivo. In one example, the transient electrode 10 completely biodegrades after a period of time in vivo.

In another embodiment, the period of time in which the transient electrode 10 biodegrades can be less than about 5 years, less than about 4 years, less than about 3 years, less than about 2 years, or less than about 1 year. In some instances, the period of time in which the transient electrode 10 biodegrades can be about 1 day to about 7 days, about 2 days to about 7 days, about 3 days to about 7 days, about 4 days to about 7 days, about 5 days to about 7 days, or about 6 days to about 7 days. In other instances, the period of time in which the transient electrode 10 biodegrades can be between about 1 to 24 hours, such as less than about 1 hour, about 1 hour, about 2-3 hours, about 3-4 hours, about 4-5 hours, about 5-6 hours, about 6-7 hours, about 7-8 hours, about 8-9 hours, about 9-10 hours, about 10-11 hours, about 11-12 hours, about 12-13 hours, about 13-14 hours, about 14-15 hours, about 15-16 hours, about 16-17 hours, about 17-18 hours, about 18-19, about 19-20 hours, about 20-21 hours, about 21-22 hours, about 22-23 hours, or about 24 hours.

In one example, the period of time in which the transient electrode 10 biodegrades can be 14 days or about 14 days.

Referring to FIGS. 1A-C, a transient electrode 10 can comprise an electrically non-conductive core substrate 12, a continuous, electrically-conductive metal layer 14 that envelops at least a portion of the core substrate, an optional interlayer 16 that is disposed between the core substrate and the metal layer to promote adhesion between the core substrate and the metal layer, and an optional barrier layer 18 that at least partially envelops the metal layer.

In one embodiment, the electrically non-conductive core substrate 12 can comprise a bioresorbable suture that is made, for example, from a material selected from the group consisting of poliglecaprone, polydioxanone, polyglactin, polyglycolic acid, polylactic acid, polyvinyl alcohol, collagen, and combinations thereof.

In one embodiment, the electrically non-conductive core substrate 12 can have a diameter of about 10 microns to about 500 microns, for example, about 50 microns, about 100 microns, about 150 microns, about 200 microns, about 250 microns, about 300 microns, about 350 microns, about 400 microns, about 450 microns, or about 500 microns. In one example, the electrically non-conductive core substrate 12 can have a diameter of about 30 microns to about 300 microns. In another example, the electrically non-conductive core substrate 12 can have a standard suture size of 9-0 to 2-0.

In one embodiment, the electrically non-conductive core substrate 12 can have an initial tensile strength (e.g., before the transient electrode 10 is implanted in vivo) of about 1 Newton to about 200 Newtons, for example, about 25 Newtons, about 50 Newtons, about 75 Newtons, about 100 Newtons, about 125 Newtons, about 150 Newtons, about 175 Newtons, or about 200 Newtons. In one example, the electrically non-conductive core substrate 12 can have a tensile strength of about 5 Newtons to about 100 Newtons.

In one embodiment, the electrically non-conductive core substrate 12 can have a tensile strength that is reduced or diminished by at least about 50% (e.g., about 55%, about 60%, about 65%, about 70%, about 75%, or about 80% or more) over a period of time after implantation of the transient electrode 10 in vivo. In one example, the period of time after implantation of the transient electrode 10 in vivo can be about 1 week to about 8 weeks, for example, about 1 week, about 2 weeks, about 3 weeks, about 4 weeks, about 5 weeks, about 6 weeks, about 7 weeks, or about 8 weeks. In one example, the electrically non-conductive core substrate 12 can have a tensile strength that is reduced or diminished by at least about 50% (e.g., about 50% or 50%) over a period of about 1-8 weeks following implantation of the transient electrode 10 in vivo.

In another embodiment, the electrically non-conductive core substrate 12 can have a glass transition temperature of about −10 to about 50 degrees Celsius. In one example, the electrically non-conductive core substrate 12 can have a glass transition temperature of about zero degrees Celsius (e.g., zero degrees Celsius). In another example, the electrically non-conductive core substrate 12 can have a glass transition temperature of greater than zero degrees Celsius.

In another embodiment, complete degradation and/or resorption of the electrically non-conductive core substrate 12, following implantation of the transient electrode 10 in vivo, can occur within about 1-12 months, for example, within about 1 month, within about 2 months, within about 3 months, within about 4 months, within about 5 months, within about 6 months, within about 7 months, within about 8 months, within about 9 months, within about 10 months, within about 11 months, or within about 12 months. In one example, complete degradation and/or resorption of the electrically non-conductive core substrate 12, following implantation of the transient electrode 10 in vivo, can occur within about 2-8 months.

In some instances, all or only a portion of a surface of the electrically non-conductive core substrate 12 can be modified or treated to improve surface area, physical properties and/or chemical properties thereof, through means including, but not limited, to chemical etching, physical abrasion, and/or plasma treatment, to improve bond strength with the metal layer 14 and/or the barrier layer 18 (see, e.g., Mozetič M., Materials (Basel). 2019 Jan. 31; 12(3):441; Gilliam, M. (2013). Polymer Surface Treatment and Coating Technologies. In: Nee, A. (eds) Handbook of Manufacturing Engineering and Technology (Springer, London); and Droździel-Jurkiewicz M. et al., Materials (Basel). 2022 Sep. 3; 15(17):6118).

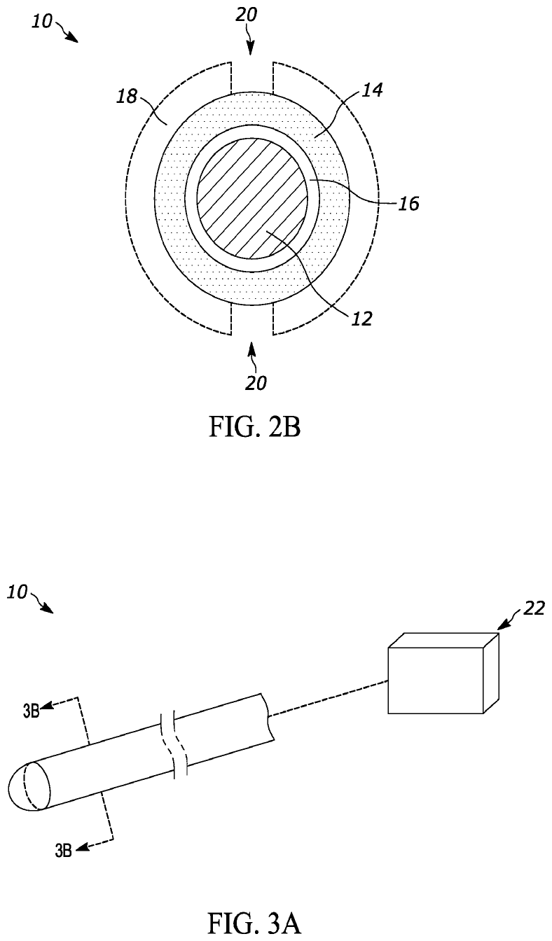

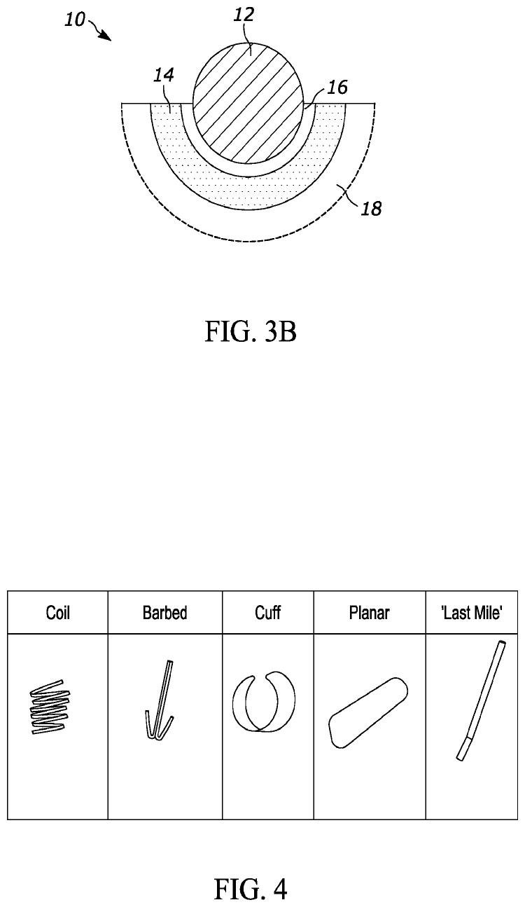

In another embodiment, the continuous, electrically-conductive metal layer 14 can envelop all or only a portion of the core substrate 12. As shown in FIGS. 1A-C and FIGS. 2A-B, for example, the electrically-conductive metal layer 14 can cover or envelop all of the core substrate 12. Alternatively, as shown in FIGS. 3A-B, the electrically-conductive metal layer 14 can cover only a portion (i.e., less than the entire) of the core substrate 12 such that a portion of the core substrate is directly exposed to the ambient environment.

In one embodiment, the metal layer 14 comprises a single (pure) bioinert material (e.g., metal) or a combination (alloyed or composite) of bioinert materials (e.g., a bimetal), such as gold, platinum, palladium, titanium, tantalum, rhodium, iridium, magnesium, zinc, iron and molybdenum, manganese, as well as oxides thereof and nitrides thereof. In another example, the metal layer 14 is pure gold.

In one embodiment, the metal layer 14 comprises a degradable metal or combination of metals. The use of a degradable metal layer for a transient electrode 10, as opposed to a metal layer that is resorbable, is advantageous because resorbable metals (e.g., magnesium) have very high impedances (e.g., greater than 1000 Ohms) relative to the transient electrode of the present disclosure and, as accordingly, use of such resorbable metals do not yield functional devices for certain neural stimulation applications. Advantageously, the metal layer 14 comprising the transient electrode 10 imparts the electrode with an impedance that is significantly lower than conventional electrodes made of resorbable metals; for example, the transient electrode of the present disclosure can have an impedance of about 2 Ohms to about 2000 Ohms, e.g., about 2 Ohms to about 50 Ohms, about 50 Ohms to about 150 Ohms, about 150 Ohms, to about 300 Ohms, about 300 Ohms to about 500 Ohms, about 500 Ohms to about 1000 Ohms, about 1000 Ohms to about 1500 Ohms, or about 1500 Ohms to about 2000 Ohms. In another example, the transient electrode 10 of the present disclosure can have an impedance of less than about 200 Ohms, e.g., less than about 150 Ohms (e.g., about 100 Ohms to about 150 Ohms). In a further example, the transient electrode 10 of the present disclosure can have an impedance of about 100 Ohms to about 110 Ohms.

In another embodiment, the metal layer 14 can have a uniform thickness or a thickness that varies across a partial or entire length of the metal layer. In one example, the metal layer 14 can have a varying or uniform thickness that is greater than about 50 nm. In another example, the metal layer 14 can have a thickness of equal to or less than about 1 μm, a thickness of about 1 μm to 10 μm, a thickness of about 10 μm to about 100 μm, a thickness of about 100 μm to about 1000 μm, or a thickness of about 1000 μm to about 10000 μm. In another example, the metal layer 14 can comprise pure gold and have a varying or uniform thickness of about 100 nm.

In another embodiment, the metal layer 14 can have an electrical conductivity (σ) of equal to or less than about 7×10⁷ S/m (at about 20° C.), e.g., equal to or less than about 6.3×10⁷ S/m (at about 20° C.). In one example, the metal layer 14 can have an electrical conductivity (σ) of equal to or greater than about 1×10⁷ S/m (at about 20° C.) but equal to or less than about 7×10⁷ S/m (at about 20° C.). In another example, the metal layer 14 can have an electrical conductivity (σ) of about 1×10⁷ S/m (at about 20° C.).

In another embodiment, all or only a portion of a surface comprising the metal layer 14 is microcracked.

In another embodiment, the interlayer 16 can be disposed between the core substrate 12 and the metal layer 14 to promote adhesion between the core substrate and the metal layer. In one example, the interlayer 16 can comprise pure titanium.

In one embodiment, the interlayer 16 can have a uniform thickness or a thickness that varies across a partial or entire length of the interlayer. In one example, the interlayer 16 can have a thickness equal to or less than about 1 μm, a thickness of about 1 μm to about 10 μm, a thickness of about 10 μm to about 100 μm, a thickness of about 100 μm to about 1000 μm, or a thickness of about 1000 μm to about 10000 μm. In another example, the interlayer 16 can comprise pure titanium and have a thickness that varies across a partial or entire length of the interlayer of about 5 nm.

As shown in FIGS. 1A-2B, the interlayer 16 can cover or envelop all of the core substrate 12. Alternatively, as shown in FIGS. 3A-B, the interlayer 16 can cover only a portion (i.e., less than the entire) of the core substrate 12 such that a portion of the core substrate is directly exposed to the ambient environment.

In another embodiment, the barrier layer 18 can entirely or only partially envelop the metal layer 14 (e.g., the barrier layer covers less than the entire metal layer). As shown in FIGS. 1A-C, for example, the barrier layer 18 can entirely cover or envelop the metal layer 14 and the interlayer 16. Alternatively, as shown in FIGS. 2A-3B, the barrier layer 18 can cover or envelop only a portion of the metal layer 14 and the interlayer 16 (i.e., the barrier layer covers less than the entirety of each of the metal layer and the interlayer. Advantageously, the barrier layer 18 can function as an insulative layer or coating to provide mechanical stability and/or protection for the metal layer 14, thereby prolonging electrical conduction for a prescribed period of operation.

In some instances, the barrier layer 18 is made of one or a combination of materials that is/are electrically-insulative, biotolerable, bioinert, biodegradable, and provide(s) mechanical stability to the metal layer 14. By “mechanical stability”, it is meant that one or more mechanical properties of the metal layer 14 is/are improved to sustain challenging environmental conditions, including but not limited to biofouling or physical abrasion, that may lead to discontinuity of metal throughout the intransient electrode 10 and/or non-uniformity delivery of electrical current. Non-limiting examples of which the barrier layer 18 can be formed include PEG, PLA, PGA, PCL, polyacetylene, polypyrrole, polythiophene, poly(3,4-ethylenedioxythiophene), graphene, and combinations thereof.

In one embodiment, the barrier layer 18 can have a uniform thickness or a thickness that varies across a partial or entire length of the barrier layer. In one example, the barrier layer 18 can have a thickness equal to or less than about 1 μm, a thickness of about 1 μm to about 10 μm, a thickness of about 10 μm to about 100 μm, a thickness of about 100 μm to about 1000 μm, or a thickness of about 1000 μm to about 10000 μm.

In another embodiment, the barrier layer 18 can comprise a material (e.g., graphene) having an electrical conductivity (σ) that exhibits little to no electrical conduction so as to allow current leakage throughout the metal layer 14. As such, the material or materials comprising the barrier layer 18 can have an electrical conductivity (σ) of equal to or less than about 1×10⁵ S/m (at about 20° C.). In one example, the material or materials comprising the barrier layer 18 can have an electrical conductivity (σ) of about 1×10⁵ S/m (at about 20° C.).

In another embodiment, illustrated in FIGS. 2A-B, the transient electrode 10 can be configured so as to include one or more discrete, geometrically-defined regions 20 that are surrounded, but not covered by, the barrier layer 18. All or some of the geometrically-defined regions 20 can have the same or different cross-sectional shape (e.g., circular, ovoid). Additionally, all or some of the geometrically-defined regions 20 can have the same or different dimensions and be spaced apart from one another by the same or different distance(s). Advantageously, the geometrically-defined regions 20 can permit targeted application of an electrical signal (or signals) to a target tissue by defining one or more discreet region(s) where the electrical signal(s) is/are applied to a target nervous tissue.

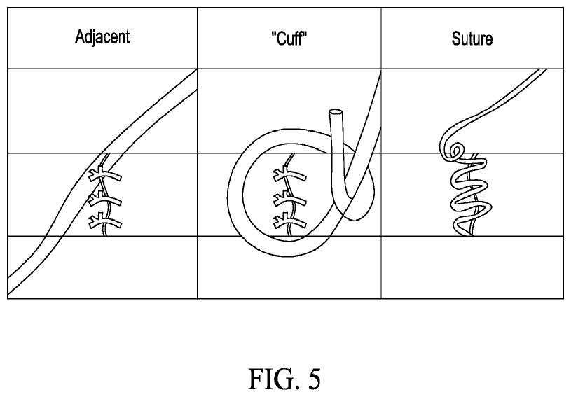

Although the transient electrode 10 of the present disclosure is illustrated herein as having an elongated, wire-like configuration, it will be appreciated that other configurations are within the scope of the present disclosure, including those illustrated in FIG. 4. In one example, the transient electrode 10 can comprise the “last mile” embodiment illustrated in FIG. 4. In this embodiment, a transient electrode 10 of the present disclosure can be sized and dimensioned for use with conventional leads, such as a percutaneous lead available from SPR Therapeutics, Inc. (Cleveland, OH). In this embodiment, the transient electrode 10 can be operably connected with a percutaneous lead 11 via an interconnect or electrical adaptor 13 (not shown in detail). Advantageously, the transient electrode 10 can interact with a target nerve and/or nearby tissue and then be left behind in vivo while the other lead components are removed from the subject. This is in contrast to conventional leads, which upon removal can break in the body and lead to metal fragments embedded into the tissue—which may or may not be biocompatible. Advantageously, the transient electrode 10 of the present disclosure is designed to fully disintegrate or degrade in the body with minimal risks to the patient. In terms of construction, fabrication methods remain the aim but interconnects/adapters may need to be developed to interface with existing lead designs.

### Systems

Another aspect of the present disclosure can include a system comprising a transient electrode 10 and a power source 22 in electrical communication with the transient electrode.

In one embodiment, the transient electrode 10 of the system can include any one or combination of the transient electrodes shown in FIGS. 1-13 and described herein.

In another embodiment, the power source 22 is capable of, or can be configured to, generate an electrical signal or signals (e.g., a therapy signal). The power source 22 can be positioned in any suitable location, such as adjacent the transient electrode 10 (e.g., implanted adjacent the transient electrode), or a remote site in or on a subject's body or away from a subject's body in a remote location. A transient electrode 10 can be connected to a remotely-positioned power source 22 using wires, e.g., which may be implanted at a site remote from the transient electrode or positioned outside the subject's body. Alternatively, the transient electrode 10 can be connected to a remotely-positioned power source 22 via a wireless connection. In one example, the power source 22 is a pulse generator (e.g., an implantable pulse generator or IPG). Other examples of suitable power sources 22 are disclosed in U.S. Pat. No. 7,483,746.

It will be appreciated that the system can include other components, such as a controller (not shown) in electrical communication with the transient electrode 10 and/or the power source 22. A controller, for example, can be configured or programmed to control the pulse waveform, the signal pulse width, the signal pulse frequency, the signal pulse phase, the signal pulse polarity, the signal pulse amplitude, the signal pulse intensity, the signal pulse duration, and combinations thereof, of a therapy signal. Further, a controller can be programmed to convey a variety of currents and voltages to one or more transient electrodes 10 of the present disclosure and, as discussed further below, thereby modulate the activity of a target nervous tissue.

### Methods of Use

Another aspect of the present disclosure can include a method for temporary neuromodulation of a target nervous tissue in a subject in need thereof. One step of the method can include advancing a transient electrode 10 of the present disclosure into electrical contact or electrical communication with the target nervous tissue. A therapy signal can then be delivered to the target nervous tissue via the transient electrode 10 for a period of time until the transient electrode completely biodegrades and is rendered non-functional.

In one embodiment, the transient electrode 10 can be advanced via a percutaneous surgical approach to the target nervous tissue. In one example, the transient electrode 10 can be advanced into electrical communication or electrical contact with one or more sensory nerves of the knee, such as a lateral superior nerve, a lateral inferior nerve, or a recurrent genicular nerve. The transient electrode 10 can be positioned about the target nervous tissue in any appropriate manner or configuration to ensure sufficient electrical communication or electrical contact with the target nervous tissue, such as those configurations shown in FIG. 5. Once the transient electrode 10 is sufficiently positioned, one or more therapy signals can be delivered to the target nervous tissue.

In one embodiment, the therapy signal can comprise one or more electrical signals configured or programmed to modulate the target nervous tissue. The therapy signal can be delivered to the transient electrode 10 either continuously, periodically, episodically, and/or a combination thereof, depending upon the condition to be treated (e.g., pain). For example, the therapy signal can be delivered in a unipolar, bipolar, and/or multipolar sequence or, alternatively, via a sequential wave, charge-balanced biphasic square wave, quasi-trapezoidal, sine wave, or any combination thereof. The therapy signal can be delivered to the transient electrode 10 all at once or, alternatively, to only a select number of regions 20 comprising the transient electrode. The particular voltage, current, and frequency of the therapy signal may be varied as needed, for example, to partially or completely disrupt action potential propagation in the target nervous tissue. Alternatively, the particular voltage, current, and frequency of the therapy signal may be varied as needed, for example, to stimulate (e.g., restore, increase or augment) action potential propagation in the target nervous tissue.

In one example, the therapy signal can be delivered to target nervous tissue in a subject suffering from post-surgical and/or post-traumatic pain.

In one embodiment, the therapy signal can be delivered to a target nervous tissue in a subject that has experienced a crushing injury, nervous tissue that has experienced a partial disruption in action potential propagation, nervous tissue that has experienced a complete disruption in action potential propagation, or a combination thereof.

In one example, target nervous tissue can include an injured nerve or nervous structure as classified by Seddon (Seddon H J, “A classification of nerve injuries”, Br Med J. 1942; 2(4260):237-9) and/or Sunderland (Sunderland S, “A classification of peripheral nerve injuries producing loss of function”, Brain. 1951; 74(4):491-516).

Another aspect of the present disclosure can include a method for temporary recording of electrical activity in a target nervous tissue in a subject. One step of the method can include advancing a transient electrode 10 of the present disclosure into electrical contact with the target nervous tissue. Using the transient electrode 10, the electrical activity of the target nervous tissue can then be recorded for a period of time until the transient electrode completely biodegrades and is rendered non-functional.

### Methods of Manufacture

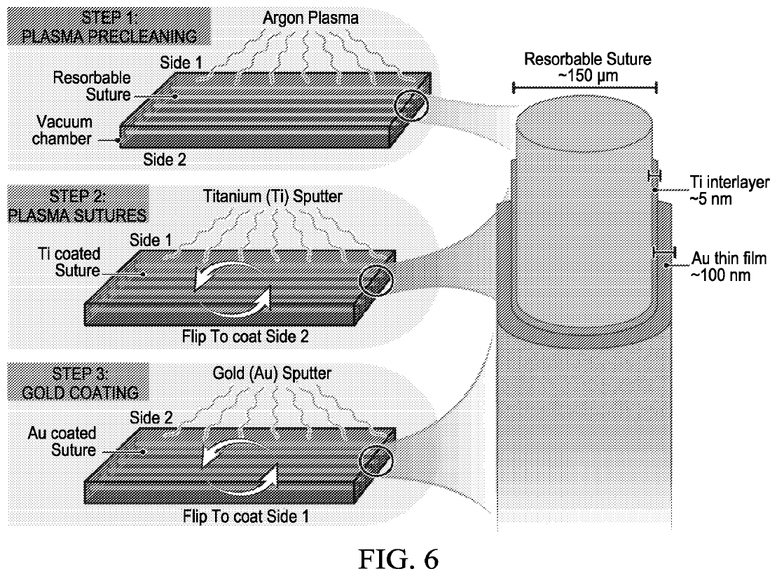

Another aspect of the present disclosure can include a method for manufacturing or fabrication of a transient electrode 10. One example of manufacturing or fabricating a transient electrode 10 is illustrated in FIG. 6 and described below.

Under clean room conditions as according to ISO 14644, the following Steps can be performed, but not necessarily in the order listed below:
Step 1. Selecting an electrically non-conductive core substrate 12, such as a bioresorbable suture;
Step 2. Lining the bioresorbable suture onto a flexible holding device; and
Step 3. Depositing one or more electrically-conductive metal layers 14 onto the bioresorbable suture by a physical vapor deposition technique or techniques (e.g., magnetron sputtering) such that the bioresorbable suture is covered in its entirety by the electrically-conductive metal layer(s).

Step 3 can comprise the following sub-steps:
a. using a holding device to place the bioresorbable suture into a deposition chamber;
b. deposition chamber is pumped down with a high vacuum, where the chamber pressure is at or below about 2×10⁶ Torr;
c. device is cleaned with an inert argon plasma for between about 30 to 60 s;
d. deposition of an interlayer (e.g., titanium) performed at a power of 250 Watts at a rate of 2.3 Å/sec;
e. deposition of an electrically-conductive metal layer (e.g., gold) performed at a power of 250 Watts at a rate of 9.7 Å/sec;
f. removal of the suture from deposition to be further modified into an active form by: addition of a barrier layer and annealing to an application-specific shape; and
g. optional addition of one or more interconnects for physical electrical connection or wireless means of electrical transfer.

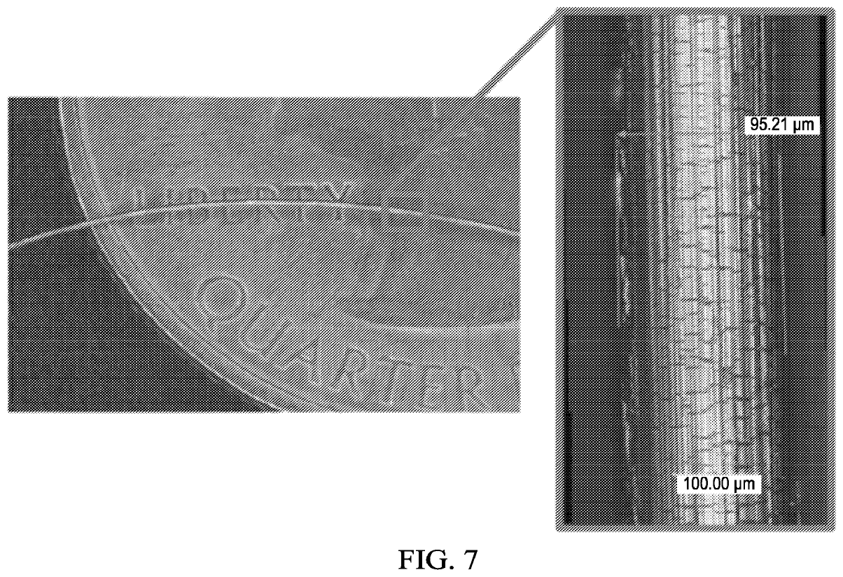

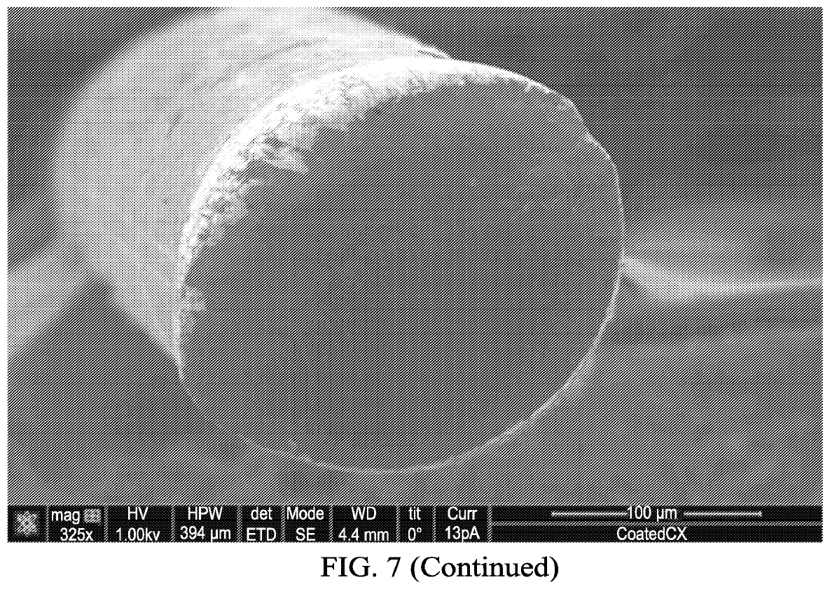

In one embodiment, a method for manufacturing or fabricating a transient electrode 10 according to the present disclosure can produce a transient electrode having a microcracked, electrically-conductive metal (gold) layer 14 as shown in FIG. 7.

### Exemplary Aspects

In view of the described compositions, devices, and methods and variations thereof, herein below are certain more particularly described aspects of the present disclosure. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A transient electrode comprising: an electrically non-conductive core substrate; a continuous, electrically-conductive metal layer that envelops at least a portion of the core substrate; an optional interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Aspect 2: A transient electrode comprising: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and an optional barrier layer that at least partially envelops the metal layer; wherein the entire electrode biodegrades and is rendered non-functional after a period of time in vivo.

Aspect 3: A transient electrode comprising: an electrically non-conductive core substrate; a continuous, electrically-conductive metal layer that envelops at least a portion of the core substrate; an interlayer that is disposed between the core substrate and the metal layer to promote adhesion between the core substrate and the metal layer; and a barrier layer that at least partially envelops the metal layer; wherein the entire electrode biodegrades and is rendered non-functional after a period of time in vivo.

Aspect 4: The transient electrode of any one of Aspects 1-3, wherein the continuous, electrically-conductive metal layer completely envelops the core substrate.

Aspect 5: The transient electrode of any one of Aspects 1-4, wherein at least a portion of a surface of the electrically non-conductive core substrate is modified or treated to improve bond strength with the metal layer and/or the barrier layer.

Aspect 6: The transient electrode of any one of Aspects 1-5, wherein the electrically non-conductive core substrate is a bioresorbable suture.

Aspect 7: The transient electrode of any one of Aspects 1-6, wherein the bioresorbable suture comprises a material selected from the group consisting of poliglecaprone, polydioxanone, polyglactin, polyglycolic acid, polylactic acid, polyvinyl alcohol, collagen, and combinations thereof.

Aspect 8: The transient electrode of any one of Aspects 1-7, wherein the metal layer comprises a bioinert material selected from the group consisting of gold, platinum, palladium, titanium, tantalum, rhodium, iridium, magnesium, zinc, iron, alloys thereof, oxides thereof, nitrides thereof, and combinations thereof.

Aspect 9: The transient electrode of any one of Aspects 1-8, wherein the interlayer comprises titanium.

Aspect 10: The transient electrode of any one of Aspects 1-9, wherein the barrier layer comprises a material selected from the group consisting of polyacetylene, polypyrrole, polythiophene, poly(3,4-ethylenedioxythiophene), graphene, and combinations thereof.

Aspect 11: The transient electrode of any one of Aspects 1-10, wherein the barrier layer is made of one or a combination of materials that is/are electrically-insulative and provide(s) mechanical stability to the metal layer.

Aspect 12: The transient electrode of any one of Aspects 1-11, wherein the barrier layer covers less than the entire metal layer.

Aspect 13: The transient electrode of any one of Aspects 1-12, including one or more discrete, geometrically-defined regions that are surrounded, but not covered by, the barrier layer.

Aspect 14: The transient electrode of any one of Aspects 1-13, wherein the period of time is less than about 5 years, between about 1 to 4 days, or about 1 to 24 hours.

Aspect 15: The transient electrode of any one of Aspects 1-14, the transient electrode of any one of claims 1-14 being configured as a percutaneous lead.

Aspect 16: The transient electrode of any one of Aspects 1-15, wherein all or only a portion of a surface comprising the metal layer is microcracked.

Aspect 17: The transient electrode of any one of Aspects 1-16, having an impedance of about 2 Ohms to about 2000 Ohms (e.g., about 2 Ohms to about 50 Ohms, about 50 Ohms to about 150 Ohms, about 150 Ohms, to about 300 Ohms, about 300 Ohms to about 500 Ohms, about 500 Ohms to about 1000 Ohms, about 1000 Ohms to about 1500 Ohms, or about 1500 Ohms to about 2000 Ohms), e.g., an impedance of less than about 200 Ohms, e.g., less than about 150 Ohms (e.g., about 100 Ohms to about 150 Ohms (e.g., about 100 Ohms to about 110 Ohms).

Aspect 18: A system comprising: the transient electrode of any one of Aspects 1-17; and a power source in electrical communication with the transient electrode.

Aspect 19: The system of Aspect 18, wherein an electrical signal is delivered from the power source to the transient electrode via one or more of a hard-wired connection, a wireless connection, capacitive coupling, and Faradaic coupling.

Aspect 20: The system of any one of Aspects 18-19, wherein the power source is a pulse generator.

Aspect 21: A method for temporary neuromodulation of a target nervous tissue in a subject in need thereof, the method comprising: advancing the transient electrode of any one of Aspects 1-17 into electrical contact with the target nervous tissue; and delivering a therapy signal to the target nervous tissue via the transient electrode for a period of time until the transient electrode completely biodegrades and is rendered non-functional.

Aspect 22: The method of Aspect 21, wherein the therapy signal is delivered to target nervous tissue that has experienced a crushing injury, nervous tissue that has experienced a partial disruption in action potential propagation, nervous tissue that has experienced a complete disruption in action potential propagation, or a combination thereof.

Aspect 23: The method of any one of Aspects 21-22, wherein the subject is suffering from post-surgical and/or post-traumatic pain.

Aspect 24: A method for temporary recording of electrical activity in a target nervous tissue in a subject, the method comprising: advancing the transient electrode of any one of Aspects 1-17 into electrical contact with the target nervous tissue; and recording, by the transient electrode, the electrical activity of the target nervous tissue for a period of time until the transient electrode completely biodegrades and is rendered non-functional.

Aspect 25: A transient electrode consisting of: a bioresorbable suture; a pure gold layer that envelops the entire bioresorbable suture, the gold layer having a thickness of about 100 nm; and a pure titanium interlayer that is disposed between the bioresorbable suture and the gold layer to promote adhesion between the bioresorbable suture and the gold layer, the titanium interlayer enveloping the entire bioresorbable suture and having a thickness of about 5 nm; wherein the entire electrode biodegrades and is rendered non-functional after a period of time in vivo.

Aspect 26: The transient electrode of Aspect 25, further comprising a barrier layer that entirely envelops the gold layer, the barrier layer comprising a material selected from the group consisting of polyacetylene, polypyrrole, polythiophene, poly(3,4-ethylenedioxythiophene), graphene, and combinations thereof.

The following Example is for the purpose of illustration only and is not intended to limit the scope of the claims, which are appended hereto.

### Example

Electrical stimulation after peripheral nerve injury (PNI) has the potential to promote more rapid and complete recovery of damaged fiber tracts. While permanently implanted devices are commonly used to treat chronic or persistent conditions, they are not ideal solutions for transient medical therapies due to high costs, increased risk of surgical injury, irritation, infection, and persistent inflammation at the site of the implant. Furthermore, removal of temporary leads placed on or around peripheral nerves may have unacceptable risk for nerve injury, which is counterproductive in developing therapies for PNI treatment. Transient devices which provide effective clinical stimulation while being capable of harmless bioabsorption may overcome key challenges in these areas. However, current bioabsorbable devices are limited in their robustness and require complex fabrication strategies and novel materials which may complicate their clinical translation pathway. In this Example, the inventors developed a transient electrode fabricated by modifying standard absorbable sutures and demonstrated surprising stability and performance in vitro and in vivo.

### Methods

### Fabrication of Transient Electrode

A synthetic bioresorbable suture, monofilament poliglecaprone (PGC, 0.1 mm diameter Monocryl) was selected in this study as a degradable backbone for the architecture (FIG. 6). To fabricate the electrode, a thin gold film was deposited onto the resorbable PGC suture substrate by DC magnetron sputtering (Discovery 18, Denton Vacuum LLC, Moorestown, NJ). Suture threads were prepared by threading plain PGC onto a flexible holding rack and securing threads by Kapton tape prior to placement in a sputtering chamber. The sputtering chamber was pumped to a background pressure of 3.0×10⁻⁷ Torr prior to introduction of 100% Ar at 2×10⁻⁶ Torr. The sample surface was roughened and decontaminated by an Ar plasma for 30 seconds prior to sputter deposition. DC power of 250 W was applied over the deposition process, first to a 99.99% pure Ti target, and then to a 99.99% pure Au target. Thin titanium and gold layers were deposited for a specific amount of time calculated from a known material deposition rate, and the sample rack in the chamber was flipped to ensure an even film coating across the sample. After deposition, threads of varying lengths were cut from the rack. Specific film thicknesses were obtained by measurement using a stylus profilometer (Tencor ε-6 Surface Profilometer, KLA, Milpitas, CA), and quality of thin film coverage was observed and quantified via digital microscope (VHX-7000, Keyence, Itasca, IL) and scanning electron microscopy (Apreo-2, Thermo-Fisher Scientific, Waltham, MA) (FIG. 7).

### Baseline Electrochemical Properties

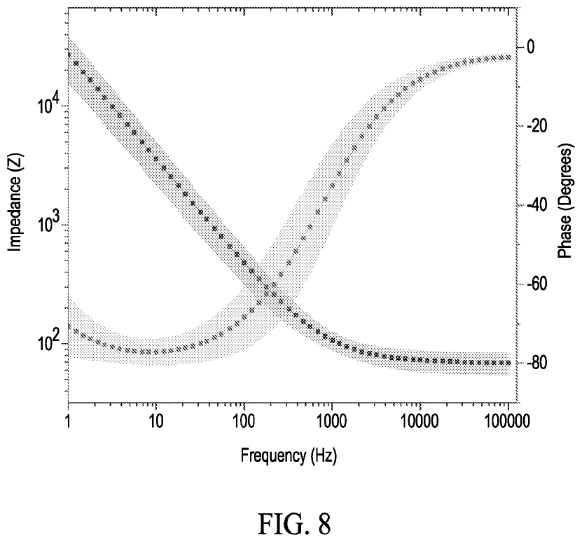

Cyclic voltammetry (CV) and electrochemical impedance spectroscopy (EIS) measurements were performed using a standard three-electrode cell consisting of our fabricated devices as the working electrode, a high surface area platinum counter electrode, and a single junction 3M KCl Ag/AgCl reference electrode in a phosphate-buffered saline (PBS) bath. Measurements were made using a potentiostat system (Interface 1010E, Gamry, Warminster, PA). Working electrodes (n=8) were fabricated by attachment of conductive suture to stainless steel wire via a conductive epoxy, and a 1 cm length of suture electrode was submerged in solution for analysis. CV sweeps (n=8) between −0.6 to 0.9 V versus Ag/AgCl were performed at a sweep rate of 50 mV/s and a step size of 2.44 mV, with the final three complete voltammograms averaged to obtain a single measurement. Baseline cathodic charge storage capacity (CSCc) was calculated as the averaged time integral of the cathodic current. EIS was performed after CV sweeps in the same cell using a 50 mV sinusoidal waveform applied from 1 Hz to 100 kHz to study baseline impedance and phase (FIG. 8).

### Degradation in Accelerated Aging Environment

An accelerated aging protocol was set up to evaluate electrode durability under simulated biological stress. In this Example, standard working electrodes (n=6) were fabricated, with the active length of conductive suture immersed in a 20 mM hydrogen peroxide in 1×PBS solution held at 37° C. Injections of concentrated hydrogen peroxide solution were repeated to maintain an appropriate concentration over the Example's duration as prescribed by Takmakov et al. (Journal of Neural Engineering, vol. 12, no. 2, 2015, p. 026003). Weekly EIS and CV measurements were performed alongside digital microscope imaging to assess impedance, charge storage capacity, and electrode structural integrity over time.

### Electrode Implant and Impedance Recordings

All animal procedures were reviewed and approved by the Case Western Reserve University IACUC. In this Example, a length of coated suture (2.0 cm) was attached to a stainless-steel wire via a physical interconnect (conductive epoxy, polyolefin tubing, medical silicone) to form the proposed stimulating nerve electrode. Fabricated electrodes (n=7, 1.5 cm active length) were implanted subcutaneously into the back (rostral) of anesthetized Sprague-Dawley rats (n=4), with stainless-steel wire leads drawn back to an Omnetics “micro” connector (MCS-16-SS, Omnetics, Minneapolis, MN) secured to the rodent skull. A stainless-steel reference electrode was placed distally in the back for passive impedance recordings at 1 kHz (LabRat LR10, Tucker-David Technologies, Alachua, FL).

### Results

### Design and Fabrication

FIG. 6 illustrates the fabrication process of the transient electrode, whereby a large and scalable quantity of conductive wires may be produced in a chamber by threading pre-synthesized absorbable suture back and forth along a holding rack, with threads interspersed several millimeters apart. Rotation of 5-0 PGC suture on the rack enabled a complete coating around the cylindrical suture using a minimal quantity of titanium (5 nm) as the primary adhesion promotor for polymer to subsequently deposited gold (100 nm), as evaluated using digital and scanning electron microscopy (FIG. 7). PGC (Monocryl) suture was evaluated in this Example as a substrate candidate. It is a segmented block copolymer of hard glycolide (75%) and soft-caprolactone segments (25%) and is available in monofilament configuration for a wide range of sizes. In selecting an initial substrate candidate from a list of multiple synthetic absorbable sutures, PGC was chosen based on its low relative tissue reaction score, pliability and ease of handling, stability under sputtering conditions, and predictable hydrolytic degradation, where the PGC substrate will resorb fully via hydrolysis in 90 to 120 days. A thin gold film was chosen as a conductor in this Example over commonly used biodegrading metals such as magnesium or iron. Though complete degradation is desirable, the rapid day-to-day corrosion of highly bioresorbable metals adds extra complexity when attempting to optimize a device for longer operational lifespans. Internal benchtop assessments (not reported in this Example) qualitatively support the use of a light titanium adhesion promotor (or interlayer) towards improving gold-on-polymer stability, reducing instances of erratic delamination after fabrication and in wetting solutions. Deposition thicknesses in the range of a few nanometers to a few hundred nanometers of gold and titanium were chosen to minimize the amount of trace ions released after comparatively rapid substrate resorption, while still maintaining electrochemical stability during operation.

### Electrochemical Impedance Spectroscopy and Cyclic Voltammetry at Baseline

Baseline EIS assessment of transient electrodes (surface area, 0.0314 cm²) show an average 106.89±19.88Ω observed at 1 KHz. The phase diagram shows standard capacitive charge transfer behavior at lower frequencies with a decreasing phase angle reflecting proportionally greater resistive behavior as frequency increases.

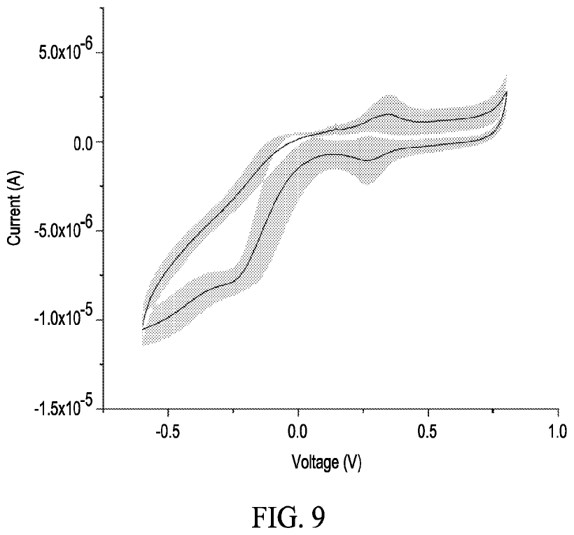

Cyclic voltammetry measurements of the transient electrodes reflect measurements of bare gold electrodes in PBS buffer. The measured electrodes have an average cathodic charge storage capacity (CSCc) of 93.01 μC/cm² at baseline (FIG. 9).

### Electrochemical Degradation Assessment

An in vitro protocol for rapid screening of implant stability was described by Takmakov et al., Reactive accelerated aging (RAA) experiments seek to create harsh environments by use of elevated temperature saline to increase the rates of degrading chemical reactions, and hydrogen peroxide to mimic the effects of in vivo oxidative stresses on an electrode. The protocol and test platform described in Takmakov et al. proposes a one-week immersion in lower (10-20 mM) H₂O₂ concentrations in PBS, coupled with very high temperatures (87° C.) to simulate aging a chronic implant by 6 months. In this Example, devices were subjected to rapid aging by 20 mM H₂O₂ in PBS at simulated body temperature (37° C.) for a longer (4-week) evaluation. High temperature RAA as performed in Takmakov et al. was further precluded due to the structural change that occurs to polymers exposed to high temperatures (>60° C.), which make relevant degradation kinetics nonlinear and unpredictable. Based on the transient electrode's much shorter (<1-month) anticipated lifespan, this accelerated aging protocol was deemed sufficient to assess the impact of the chemical environment on electrode stability.

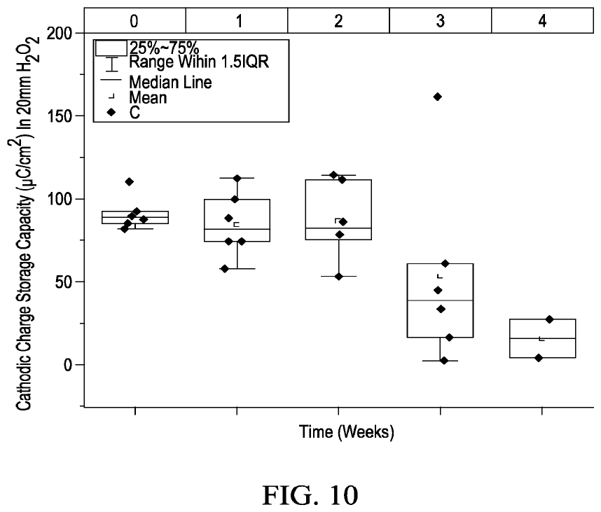

Measurements revealed distinct changes in EIS and CV behavior which corresponded to the relative degree of abiotic failure, or implant degradation. These changes are observed through microscopy and quantified through impedance measurements at 1 KHz and CSCc over time. Since the electrodes are meant to fail over time by design, here we evaluated CSCc not as a strict determination of total reversible charge injection capacity, which we expect to resemble pure gold at baseline, but rather as an indication of electrode performance stability over time. In the first two weeks, there were no significant deviations in recorded mean (109.84±10.82Ω) or median (105.18Ω) impedance. CSCc and variance in CSCc increases marginally between the baseline (93.61±11.52 μC/cm²) and second week measurement (97.11±18.88 μC/cm²) (FIG. 10).

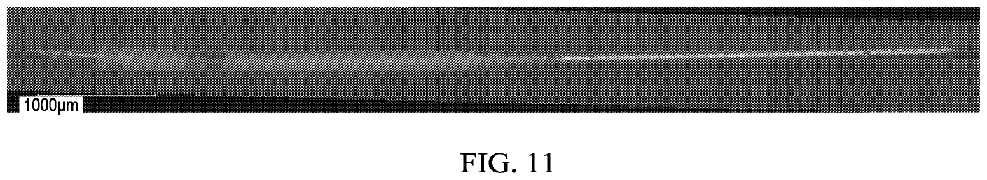

Under microscopy, evidence of the beginnings of delamination and slight substrate swelling become more apparent (FIG. 11).

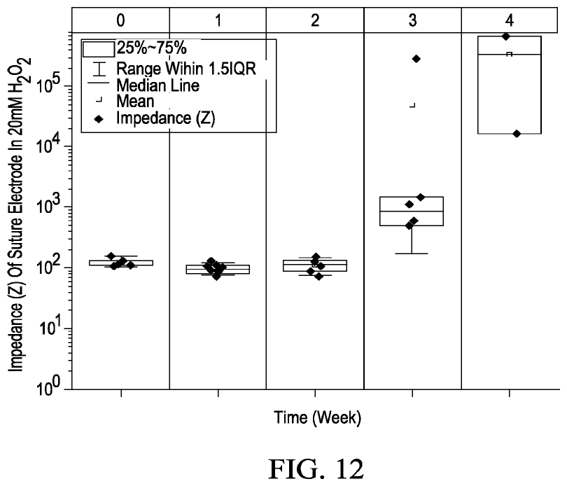

By Week 3, median impedance increases by a factor of 5.4×, while median charge storage capacity decreases by a factor of 2.5× (FIG. 12). Imaging at week four revealed several electrodes to have shortened, with distal ends partially dissolved into solution.

Results of this preliminary analysis suggest that a transient electrode will fail completely within 2 to 3 weeks by substrate swelling and subsequent thin-film delamination, with significant substrate dissolution visible by week four.

### Passive Suture Electrode Survival In Vivo

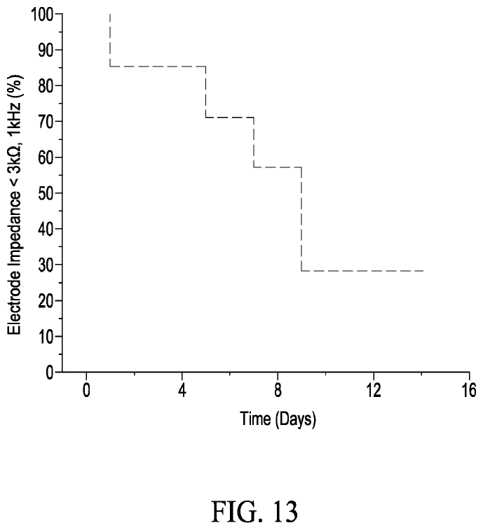

To validate the results of our in vitro RAA assessment, transient electrodes were implanted subcutaneously, and impedances observed over 14 days. Within 9 days, only two electrodes remained viable with impedances below 3 kΩ (impedance at 1 KHz was chosen as a convenient benchmark) (FIG. 13).

From the above description of the present disclosure, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

1. A transient electrode comprising:
an electrically non-conductive core substrate;
a continuous, electrically-conductive metal layer that envelops at least a portion of the core substrate;
an optional interlayer that is disposed between the core substrate and the metal layer to promote adhesion between the core substrate and the metal layer; and
an optional barrier layer that at least partially envelops the metal layer;
wherein the entire electrode biodegrades and is rendered non-functional after a period of time in vivo.

2. A transient electrode comprising:
an electrically non-conductive core substrate;
a continuous, electrically-conductive metal layer that envelops at least a portion of the core substrate;
an interlayer that is disposed between the core substrate and the metal layer to promote adhesion between the core substrate and the metal layer; and
an optional barrier layer that at least partially envelops the metal layer;
wherein the entire electrode biodegrades and is rendered non-functional after a period of time in vivo.

3. A transient electrode comprising:
an electrically non-conductive core substrate;
a continuous, electrically-conductive metal layer that envelops at least a portion of the core substrate;
an interlayer that is disposed between the core substrate and the metal layer to promote adhesion between the core substrate and the metal layer; and
a barrier layer that at least partially envelops the metal layer;
wherein the entire electrode biodegrades and is rendered non-functional after a period of time in vivo.

4. The transient electrode of any one of claims 1-3, wherein the continuous, electrically-conductive metal layer completely envelops the core substrate.

5. The transient electrode of any one of claims 1-4, wherein at least a portion of a surface of the electrically non-conductive core substrate is modified or treated to improve bond strength with the metal layer and/or the barrier layer.

6. The transient electrode of any one of claims 1-5, wherein the electrically non-conductive core substrate is a bioresorbable suture.

7. The transient electrode of any one of claims 1-6, wherein the bioresorbable suture comprises a material selected from the group consisting of poliglecaprone, polydioxanone, polyglactin, polyglycolic acid, polylactic acid, polyvinyl alcohol, collagen, and combinations thereof.

8. The transient electrode of any one of claims 1-7, wherein the metal layer comprises a bioinert material selected from the group consisting of gold, platinum, palladium, titanium, tantalum, rhodium, iridium, magnesium, zinc, iron, alloys thereof, oxides thereof, nitrides thereof, and combinations thereof.

9. The transient electrode of any one of claims 1-8, wherein the interlayer comprises titanium.

10. The transient electrode of any one of claims 1-9, wherein the barrier layer comprises a material selected from the group consisting of polyacetylene, polypyrrole, polythiophene, poly(3,4-ethylenedioxythiophene), graphene, and combinations thereof.

11. The transient electrode of any one of claims 1-10, wherein the barrier layer is made of one or a combination of materials that is/are electrically-insulative and provide(s) mechanical stability to the metal layer.

12. The transient electrode of any one of claims 1-11, wherein the barrier layer covers less than the entire metal layer.

13. The transient electrode of any one of claims 1-12, including one or more discrete, geometrically-defined regions that are surrounded, but not covered by, the barrier layer.

14. The transient electrode of any one of claims 1-13, wherein the period of time is less than about 5 years, between about 1 to 4 days, or about 1 to 24 hours.

15. The transient electrode of any one of claims 1-14 being configured as a percutaneous lead.

16. The transient electrode of any one of claims 1-15, wherein all or only a portion of a surface of the metal layer is microcracked.

17. The transient electrode of any one of claims 1-16, having an impedance of about 2 Ohms to about 2000 Ohms.

18. The transient electrode of any one of claims 1-17, having an impedance of less than about 200 Ohms.

19. A system comprising:
the transient electrode of any one of claims 1-18; and
a power source in electrical communication with the transient electrode.

20. The system of claim 19, wherein an electrical signal is delivered from the power source to the transient electrode via one or more of a hard-wired connection, a wireless connection, capacitive coupling, and Faradaic coupling.

21. The system of any one of claims 19-20, wherein the power source is a pulse generator.

22. A method for temporary neuromodulation of a target nervous tissue in a subject in need thereof, the method comprising:
advancing the transient electrode of any one of claims 1-18 into electrical contact with the target nervous tissue; and
delivering a therapy signal to the target nervous tissue via the transient electrode for a period of time until the transient electrode completely biodegrades and is rendered non-functional.

23. The method of claim 22, wherein the therapy signal is delivered to target nervous tissue that has experienced a crushing injury, nervous tissue that has experienced a partial disruption in action potential propagation, nervous tissue that has experienced a complete disruption in action potential propagation, or a combination thereof.

24. The method of any one of claims 22-23, wherein the subject is suffering from post-surgical and/or post-traumatic pain.

25. A method for temporary recording of electrical activity in a target nervous tissue in a subject, the method comprising:
advancing the transient electrode of any one of claims 1-18 into electrical contact with the target nervous tissue; and
recording, by the transient electrode, the electrical activity of the target nervous tissue for a period of time until the transient electrode completely biodegrades and is rendered non-functional.
